(19) United States
(12) Patent Application Publication
(10) Pub. No.: US 2017/0095366 A1
(43) Pub. Date: Apr. 6, 2017

(54) APPARATUS AND METHOD FOR A POCKET SLING JERSEY

(71) Applicants: Todd Eric Thrasher, Chattanooga, TN (US); Miranda O'Shields Burt, Rossville, GA (US)

(72) Inventors: Todd Eric Thrasher, Chattanooga, TN (US); Miranda O'Shields Burt, Rossville, GA (US)

(21) Appl. No.: 15/286,119

(22) Filed: Oct. 5, 2016

Related U.S. Application Data
(60) Provisional application No. 62/237,218, filed on Oct. 5, 2015.

(51) Int. Cl. A61F 5/37; A41D 27/20; A41D 1/04

(52) U.S. Cl. CPC A61F 5/3746; A41D 1/04; A41D 27/20; A61F 5/373; A41D 2400/60

(57) ABSTRACT

A jersey adapted to be worn on a user's upper body comprising a torso portion having a first opening adjacent to the user's waist, a second opening adjacent to the user's neck, a third opening adjacent to the user's left arm, and a fourth opening adjacent to the user's right arm. The jersey also comprises a pocket sling disposed on the torso portion of the jersey and defining in part a hand opening and an arm opening. The pocket sling is adapted to receive and removably retain the user's arm. A method for stabilizing a user's arm and shoulder comprising providing a jersey, wearing the jersey on the user's upper body, and removably retaining the user's arm in the pocket sling.

### CROSS-REFERENCES TO RELATED APPLICATIONS/PATENTS

This application relates back to and claims the benefit of priority from U.S. Provisional Application for Patent Ser. No. 62/237,218 titled “Arm Sling Shirt” and filed on Oct. 5, 2015.

### FIELD OF THE INVENTION

The present invention relates generally to apparel, and particularly to medical apparel.

### BACKGROUND AND DESCRIPTION OF THE PRIOR ART

It is known to use an arm sling to support an injured or disable arm. Conventional arm slings, however, suffer from one or more disadvantages. For example, conventional arm slings do not provide the user's arm with adequate support, stability, and immobility. In addition, conventional arm slings do not provide the user's shoulders with adequate support, stability, and immobility. Indeed, conventional arm slings cause the user's injured shoulder to drop which prevents the injured shoulder from healing properly. Conventional arm slings also cause discomfort and irritation to the user's arm, neck and shoulders. Conventional arm slings require additional straps in order to immobilize the user's arm. Conventional arm slings are undesirably thick, they do not minimize the user's perspiration, and they do not maximize the weight distribution on the user's arm.

It would be desirable, therefore, if an apparatus and method for a pocket sling jersey could be provided that would provide the user's arm with adequate support, stability, and immobility. It would also be desirable if such an apparatus and method for a pocket sling jersey could be provided that would provide the user's shoulders with adequate support, stability, and immobility. It would be further desirable if such an apparatus and method for a pocket sling jersey could be provided that would prevent the user's injured shoulder from dropping and as a result prevent the injured shoulder from healing properly. It would be still further desirable if such an apparatus and method for a pocket sling jersey could be provided that would minimize or eliminate discomfort and irritation to the user's arm, neck and shoulders. It would also be desirable if such an apparatus and method for a pocket sling jersey could be provided that would not require additional straps in order to immobilize the user's arm. In addition, it would be desirable if such an apparatus and method for a pocket sling jersey could be provided that would not be undesirably thick, that would minimize the user's perspiration, and that would maximize the weight distribution on the user's arm.

### ADVANTAGES OF THE PREFERRED EMBODIMENTS OF THE INVENTION

Accordingly, it is an advantage of the preferred embodiments of the invention claimed herein to provide an apparatus and method for a pocket sling jersey that provide the user's arm with adequate support, stability, and immobility. It is also an advantage of the preferred embodiments of the invention claimed herein to provide an apparatus and method for a pocket sling jersey that provide the user's shoulders with adequate support, stability, and immobility. It is another advantage of the preferred embodiments of the invention claimed herein to provide an apparatus and method for a pocket sling jersey that prevent the user's injured shoulder from dropping and as a result prevent the injured shoulder from healing properly. It is a further advantage of the preferred embodiments of the invention claimed herein to provide an apparatus and method for a pocket sling jersey that minimize or eliminate discomfort and irritation to the user's arm, neck and shoulders. It is a still further advantage of the preferred embodiments of the invention claimed herein to provide an apparatus and method for a pocket sling jersey that does not require additional straps in order to immobilize the user's arm. It is yet another advantage of the preferred embodiments of the invention claimed herein to provide an apparatus and method for a pocket sling jersey that is not undesirably thick, that minimize the user's perspiration, and that maximize the weight distribution on the user's arm.

Additional advantages of the preferred embodiments of the invention will become apparent from an examination of the drawings and the ensuing description.

### SUMMARY OF THE INVENTION

The apparatus of the invention comprises a jersey adapted to be worn on a user's upper body. The preferred jersey comprises a torso portion having a first opening adjacent to the user's waist, a second opening adjacent to the user's neck, a third opening adjacent to the user's left arm, and a fourth opening adjacent to the user's right arm. The preferred jersey also comprises a pocket sling which is disposed on the torso portion of the jersey and defines in part a hand opening and an arm opening. The preferred pocket sling is adapted to receive and removably retain the user's arm.

The method of the invention comprises a method for stabilizing a user's arm and shoulder. The preferred method comprises providing a jersey adapted for use on the user's upper body. The preferred jersey comprises a torso portion having a first opening adjacent to the user's waist, a second opening adjacent to the user's neck, a third opening adjacent to the user's left arm, and a fourth opening adjacent to the user's right arm. The preferred jersey also comprises a pocket sling which is disposed on the torso portion of the jersey and defines in part a hand opening and an arm opening. The preferred pocket sling is adapted to receive and removably retain the user's arm. The preferred method also comprises wearing the jersey on the user's upper body and removably retaining the user's arm in the pocket sling.

### BRIEF DESCRIPTION OF THE DRAWINGS

The presently preferred embodiments of the invention are illustrated in the accompanying drawings, in which like reference numerals represent like parts throughout, and in which:

FIG. 1 is a front view of the preferred embodiment of the pocket sling jersey in accordance with the present invention.

### DESCRIPTION OF THE PREFERRED EMBODIMENTS OF THE INVENTION

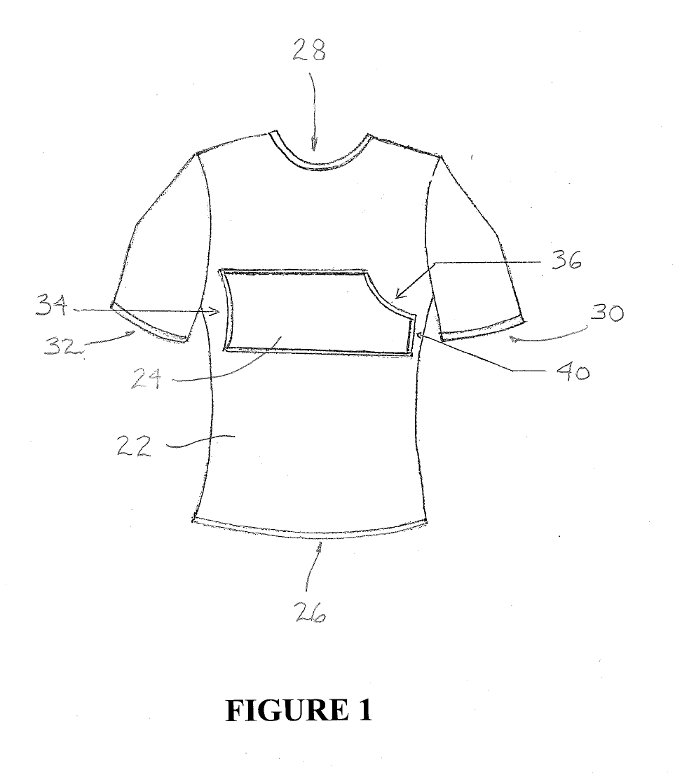

Referring now to the drawings, the preferred embodiment of the pocket sling jersey in accordance with the present invention is illustrated by FIG. 1. As shown in FIG. 1, the preferred pocket sling jersey is adapted to provide both the user's arm and shoulder with adequate support, stability, and immobility. Indeed, the preferred pocket sling jersey prevents the user's injured shoulder from dropping, and as a result, it allows the injured shoulder to heal properly. The preferred pocket sling jersey minimizes or eliminates discomfort and irritation to the user's arm, neck and shoulders. The preferred pocket sling jersey does not require additional straps in order to immobilize the user's arm. The preferred pocket sling jersey is not undesirably thick, minimizes the user's perspiration, and maximizes the weight distribution on the user's arm.

Referring now to FIG. 1, a front view of the preferred embodiment of the pocket sling jersey in accordance with the present invention is illustrated. As shown in FIG. 1, the preferred pocket sling jersey is designated generally by reference numeral 20. Preferred pocket sling jersey 20 is adapted to be worn on a user's upper body and comprises torso portion 22 and pocket sling 24. Preferred torso portion 22 comprises first opening 26 which is adjacent to the user's waist, second opening 28 which is adjacent to the user's neck, third opening 30 which adjacent to the user's left arm, and fourth opening 32 which is adjacent to the user's right arm. Preferably, torso portion 22 comprises a compression fit material and a moisture wicking material. While FIG. 1 illustrates the preferred configuration and arrangement of the torso portion of the pocket sleeve jersey, it is contemplated within the scope of the invention that the torso portion may be of any suitable configuration and arrangement. For example, it is contemplated within the scope of the invention that the torso portion may include long sleeves or no sleeves. It is also contemplated within the scope of the invention that the torso portion may include one or more zippers, buttons, snaps, hook and loop fasteners, and the like.

Still referring to FIG. 1, preferred pocket sling 24 is disposed on torso portion 22 and defines in part hand opening 34 and arm opening 36. Preferred pocket sling 24 is adapted to receive and removably retain the user's arm and comprises a compression fit material and a moisture wicking material. Preferably, pocket sling 24 evenly distributes the weight of the user's arm and substantially immobilizes the user's arm. Preferred pocket sling 24 further comprises elbow pocket 40 which is adapted to receive and removably retain the user's elbow. Preferred elbow pocket 40 extends to a location on the user's upper arm and is adapted to resist horizontal movement of the user's forearm. While FIG. 1 illustrates the preferred configuration and arrangement of the pocket sling, it is contemplated within the scope of the invention that the pocket sling may be of any suitable configuration and arrangement. For example, it is contemplated within the scope of the invention that the pocket sling may be disposed at a variety locations on the torso portion. It is also contemplated within the scope of the invention that the pocket sling may include one or more zippers, buttons, snaps, hook and loop fasteners, and the like.

The invention also comprises a method for stabilizing a user's arm and shoulder. The preferred method comprises providing a jersey as hereinabove described. The preferred method further comprises wearing the jersey on the user's upper body and removably retaining the user's arm in the pocket sling.

In operation, several advantages of the preferred embodiments of the pocket sling jersey are achieved. For example, the preferred embodiments of the pocket sling jersey provide both the user's arm and shoulder with adequate support, stability, and immobility. Indeed, the preferred pocket sling jersey prevents the user's injured shoulder from dropping, and as a result, it allows the injured shoulder to heal properly. The preferred pocket sling jersey minimizes, if not eliminates, discomfort and irritation to the user's arm, neck and shoulders. The preferred pocket sling jersey immobilizes the user's arm without any additional straps. The compression fit material of the preferred pocket sling jersey is thin and lightweight and maximizes the weight distribution on the user's arm. The moisture wicking material of the preferred pocket sling jersey minimizes the user's perspiration.

Although this description contains many specifics, these should not be construed as limiting the scope of the invention but as merely providing illustrations of some of the presently preferred embodiments thereof, as well as the best mode contemplated by the inventors of carrying out the invention. The invention, as described herein, is susceptible to various modifications and adaptations, and the same are intended to be comprehended within the meaning and range of equivalents of the appended claims.

What is claimed is:

1. A jersey adapted to be worn on a user's upper body, said jersey comprising:
(a) a torso portion, said torso portion have a first opening adjacent to the user's waist, a second opening adjacent to the user's neck, a third opening adjacent to the user's left arm, and a fourth opening adjacent to the user's right arm;
(b) a pocket sling, said pocket sling being disposed on the torso portion of the jersey and defining in part a hand opening and an arm opening;
wherein the pocket sling is adapted to receive and removably retain the user's arm.

2. The jersey of claim 1 wherein the torso portion comprises a compression fit material.

3. The jersey of claim 1 wherein the torso portion comprises a moisture wicking material.

4. The jersey of claim 1 wherein the pocket sling comprises a compression fit material.

5. The jersey of claim 1 wherein the pocket sling comprises a moisture wicking material.

6. The jersey of claim 1 wherein the pocket sling evenly distributes the weight of the user's arm.

7. The jersey of claim 1 wherein the pocket sling substantially immobilizes the user's arm.

8. The jersey of claim 1 wherein the pocket sling comprises an elbow pocket adapted to receive and removably retain the user's elbow.

9. The jersey of claim 8 wherein the elbow pocket extends to a location on the user's upper arm.

10. The jersey of claim 8 wherein the elbow pocket is adapted to resist horizontal movement of the user's forearm.

11. A method for stabilizing a user's arm and shoulder, said method comprising:
(a) providing a jersey adapted for use on the user's upper body, said jersey comprising:
(i) a torso portion, said torso portion have a first opening adjacent to the user's waist, a second opening adjacent to the user's neck, a third opening adjacent to the user's left arm, and a fourth opening adjacent to the user's right arm;
(ii) a pocket sling, said pocket sling being disposed on the torso portion of the jersey and defining in part a hand opening and an arm opening;
wherein the pocket sling is adapted to receive and removably retain the user's arm;
(b) wearing the jersey on the user's upper body; and
(c) removably retaining the user's arm in the pocket sling.

12. The method of claim 11 wherein the torso portion comprises a compression fit material.

13. The method of claim 11 wherein the torso portion comprises a moisture wicking material.

14. The method of claim 11 wherein the pocket sling comprises a compression fit material.

15. The method of claim 11 wherein the pocket sling comprises a moisture wicking material.

16. The method of claim 11 wherein the pocket sling evenly distributes the weight of the user's arm.

17. The method of claim 11 wherein the pocket sling substantially immobilizes the user's arm.

18. The method of claim 11 wherein the pocket sling comprises an elbow pocket adapted to receive and removably retain the user's elbow.

19. The jersey of claim 18 wherein the elbow pocket extends to a location on the user's upper arm.

20. The method of claim 18 wherein the elbow pocket is adapted to resist horizontal movement of the user's forearm.
